(19) United States
(12) Patent Application Publication
(10) Pub. No.: US 2005/0053533 A1
(43) Pub. Date: Mar. 10, 2005

(54) Transportable mass personnel decontamination system

(76) Inventor: Louise Brown, North Bay Ontario (CA)

Correspondence Address: SHAPIRO COHEN, P.O. BOX 3440, STATION D, OTTAWA, ON, K1P6P1, CA

(21) Appl. No.: 10/496,515

(22) PCT Filed: Nov. 25, 2002
(86) PCT No.: PCT/CA02/01792

Related U.S. Application Data
(60) Provisional application No. 60/332,565, filed on Nov. 26, 2001.

(51) Int. Cl. B08B 3/00; A61M 1/00

(52) U.S. Cl. 422/255; 134/61; 604/322

(57) ABSTRACT

The present invention provides decontamination equipment which can be on site with the first responders, and set up on the clean line, ready to process personnel as they move from the contaminated area. In one embodiment the present invention provides a transportable mass personnel decontamination system comprising a transportation means (10) carrying a plurality of individual three compartment processing cubicles (36, 39, 42); (37, 40, 43); (38, 41, 44) for ambulatory personnel, each processing cubicle having enclosure means (49, 50, 51) for each compartment to provide modesty screening of personnel, the first compartment (30, 31) for disrobing, the second compartment (32, 33) containing a temperature and pressure controlled personnel operated shower with a decontaminant dispenser, and the third compartment (34, 35) providing a re-dress area. A soft conveyor liter patient processing line (4, 5, 7) including stations for decontaminating, washing, rinsing, and drying litter patients is included. A monitoring area can be available beyond the processing cubicles. The system further includes a fresh water supply (48), and a grey water collection and storage tank (47). The fresh water system includes a temperature and pressure control (46) which specifically has an anti-scald control.

### BACKGROUND OF THE INVENTION

The present invention relates to a mass decontamination system which is readily transportable, and can be used by first responders. In the event of a natural disaster, a terrorist attack, a chemical spill or a fire, first responders, including fire fighters, police or military are dispatched to the event to rescue survivors and remove the deceased. Their roles include extinguishing fires, discerning dangerous substances, preventing infection, notifying authorities of hazardous materials including solids, liquids, gases, radiation or organisms, and cordoning off the toxic or contaminated area.

Survivors may be injured, either unable to walk, or ambulatory. Both may require decontamination. When the contaminated area has been defined, a line is established where contaminated personnel, basically everyone within the contaminated area, is required to be decontaminated before crossing the clean line. This includes first responders and survivors. Depending on the nature of the contaminant, medical personnel may be required to administer antidotes, or antibiotics. It is important that medical treatment be administered to patients who will not contaminate the medical staff. On site rapid decontamination of casualties and first responders is essential.

Transportable decontamination equipment is required which can be moved to the clean line with the first response, and set up and commence decontamination quickly.

### STATEMENT OF THE INVENTION

The present invention provides decontamination equipment which can be on site with the first responders, and set up on the clean line, ready to process personnel as they move from the contaminated area. In one embodiment the present invention provides a transportable mass personnel decontamination system comprising a transportation means carrying at least one individual three compartment processing cubicles for ambulatory personnel, each processing cubicle having enclosure means for each compartment to provide modesty screening of personnel, the first compartment for disrobing, the second compartment containing a temperature and pressure controlled personnel operated shower with a decontaminant dispenser, and the third compartment providing a re-dress area. A soft conveyor litter patient processing line including stations for decontaminating, washing, rinsing, and drying litter patients can also be included. A monitoring area can be available beyond the processing cubicles. The system further includes a fresh water supply, and a grey water collection and storage tank. The fresh water system includes a temperature and pressure control which specifically has an anti-scald control.

The system may readily transported by a road trailer, a semi-trailer, or may be contained on fork lift pallets or in an ISO shipping container, or an air cargo container, depending on the distance to be transported or the availability of transportation vehicles. In many instances the system could be trailer mounted and drawn to the site by the first responder vehicles. The trailer mounted systems can be positioned on the clean line, and so arranged that all personnel leaving the site do so through the decontamination system.

In one aspect a trailer with droppable sides forming ramps when down, contains at least one transversely mounted parallel processing cubicle, with personnel entering on one side of the trailer, disrobing, showering, and redressing, and leaving the trailer on the other side. By positioning the trailer on the clean line an efficient use is made of this configuration. The grey water system storage tank will be maintained in the contaminated area.

An adjunct to the system is a kit containing personal care and providing traceability for a victim. Such a kit includes resealable personal effect bags, disposable towels, a redress gown, slippers, and an identification wristband coded to the personal effects bags.

### BRIEF DESCRIPTION OF THE DRAWINGS

FIG. 1 is a side elevation of a trailer mounted decontamination system in road configuration,

FIG. 2 is a top view of the trailer of FIG. 1 partially prepared for use,

FIG. 3 is a plan view of the, trailer of FIGS. 1 and 2 with the top removed to show the internal arrangement,

FIG. 4 is a plan view of a decontamination site with the system fully deployed,

FIG. 5 is a plan view of an alternative embodiment,

FIG. 6 is a photograph of an alternative embodiment mounted on a cart,

FIG. 7 is a photograph of a partially erected system of FIG. 6,

FIG. 8 is a photograph of the partially assembled shower system of FIG. 6, and

FIG. 9 is a photograph of the interior of another embodiment.

### DETAILED DESCRIPTION OF THE EMBODIMENTS OF THE INVENTION

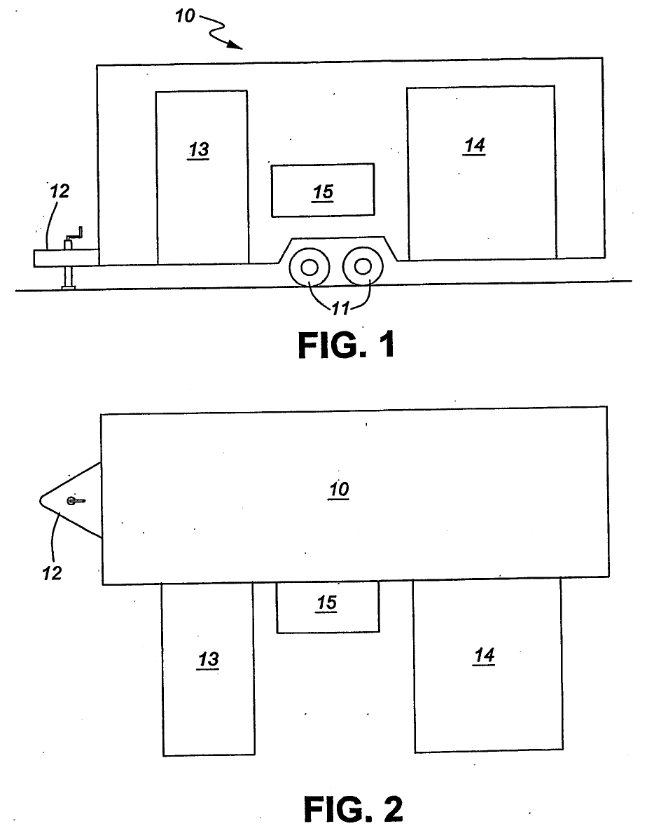

Referring to FIG. 1 there is shown a trailer mounted system of the present invention, in road configuration. The trailer 10 is mounted on a dual axle wheel system 11 and may be connected to the towing vehicle by the usual trailer hitch 12. Drop down doors 13, 14, and 15 are positioned in the sides of the trailer 10 for conversion of the trailer 10 to a decontamination station.

FIG. 2 shows the trailer 10 from above with the drop down doors extended on one side of the trailer 10. Similar doors (not shown) are located across the trailer 10 and doors 13 and 14 form ramps for ambulatory casualties. The doors 15 on either side of the trailer 10 permit a roller conveyor to be positioned through the opening formed when these doors are open.

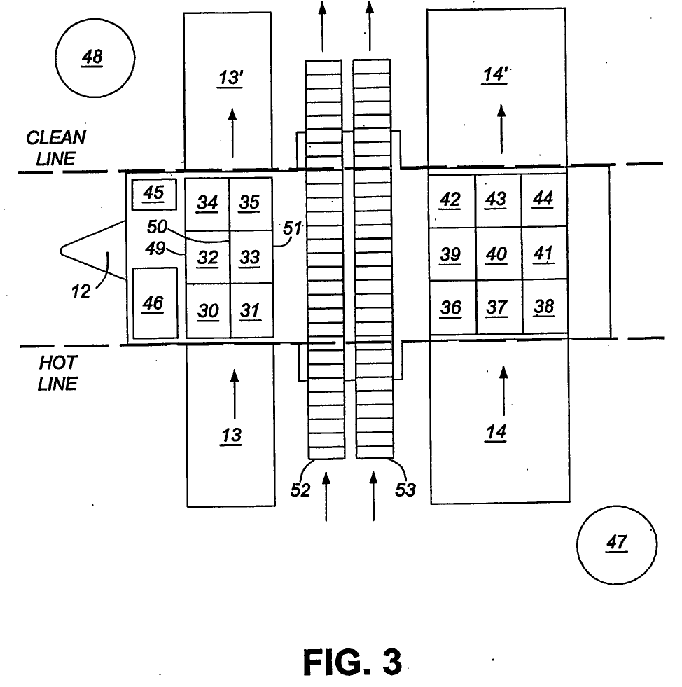

FIG. 3 shows a plan view of a decontamination system of the present invention with the top of the trailer removed to show the internal details. Ramps 13 and 13′ provide a path through processing cubicles composed of disrobing compartments 30, and 31, shower compartments 32 and 33 and drying/re-dress compartments 34 and 35.vertical walls 49, 50, and 51 made of opaque plastic material separate the cubicles and provide modesty protection for users. There are four sets of opaque plastic doors for each cubicle, separating the compartments and providing privacy for the users. Litter patients are transferred to the soft roller conveyors 52 and 53 for decontamination, washing and rinsing, drying and egress from the trailer. Such patients may have to handled unclothed, and in such a case, curtains can be arranged about the ends of the conveyors to permit these casualties to be screened from view until they have been appropriately covered. A further three sets of cubicles are provided at the rear of the trailer and are shown as cubicles 36, 39,and 42; 37, 40, and 43; and 38, 41, and 44. Thus five lines of cubicles and two litter conveyors are provided in a trailer capable of being towed by a light truck.

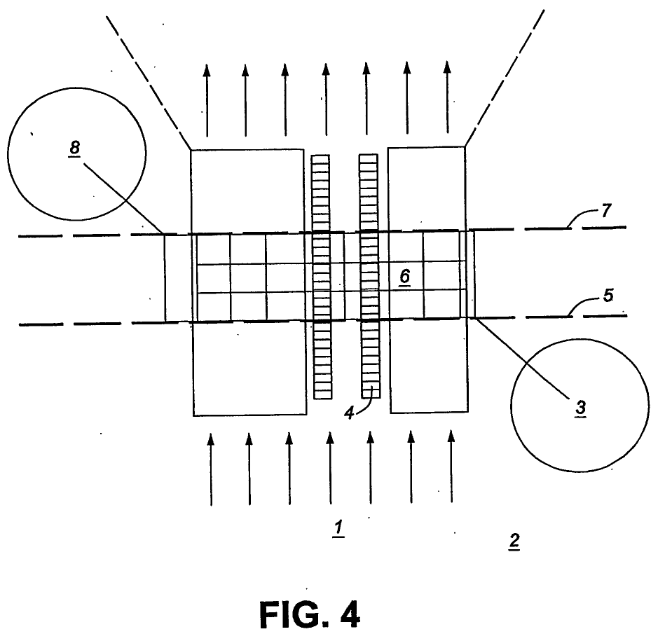

Clean water tank 48 is shown which is connected by hoses (not shown) to the water pumping and heating unit 46 and the grey water tank 47 is connected to the drain system of the showers. The Clean and Hot lines are shown in phantom in FIG. 3, the grey water tank 47 being kept in the contaminated zone defined by the Hot line and the sealed fresh water tank 48 being kept in the clean zone. The tank 47 is preferably an onion bladder type of tank, and the fresh water tank 48 is a sealed clean water collapsible NBC pillow tank. Fresh water can be replenished from a safe water source, and the grey water can be pumped from the onion bladder for disposal FIG. 4 is a plan view of the system of FIG. 3, the reference numeral 1 indicates the flow of casualties into the decontamination system, guided by control barriers 2. Reference numeral 4 indicates the grey water tank positioned in the contaminated area. Conveyors 4 carry litter patients through the system from the hot line 5 to the clean line 7. Ambulatory casualties are processed in the shower suite 6. Clean warm water is provided, to the shower suite 6 from the sealed fresh water tank 8 by the pumping and heating system described above. After the casualties have been decontaminated, they leave the system at 9.

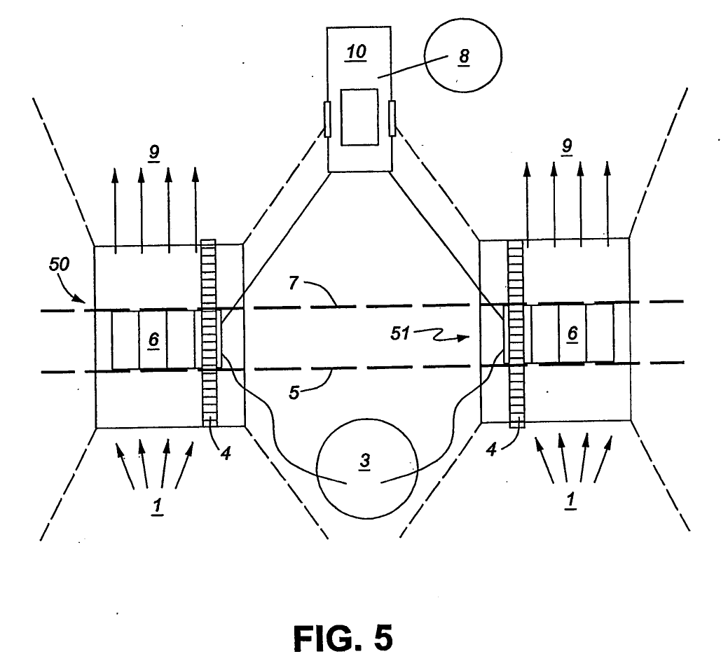

FIG. 5 is a system similar to FIG. 4, consisting of two free standing sections 50 and 51, each having a shower suite 6 and litter casualty roller systems 4. As before, sealed fresh water tank 8 provides water for the showers, and grey water tank 3 collects the drainage from the showers. A trailer 10 carries all components of the decontamination system, with appropriate connections from the trailer 10 to the free standing sections. As required the sections may be erected in Quanset or standard frame soft sided shelters, or air inflated Zumro™ shelter systems.

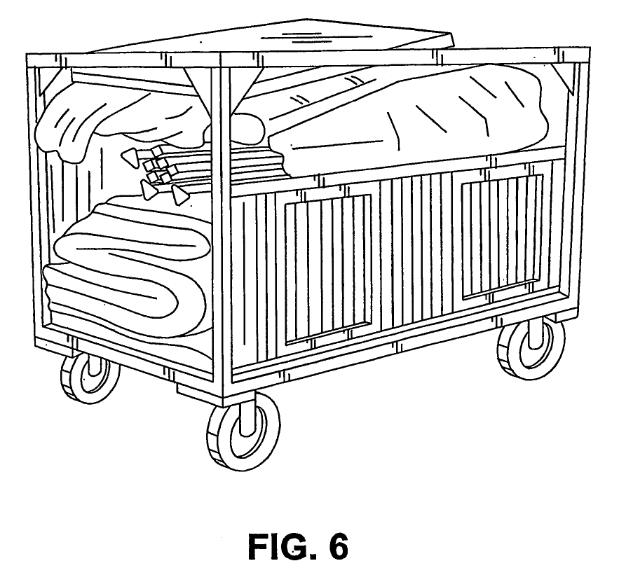

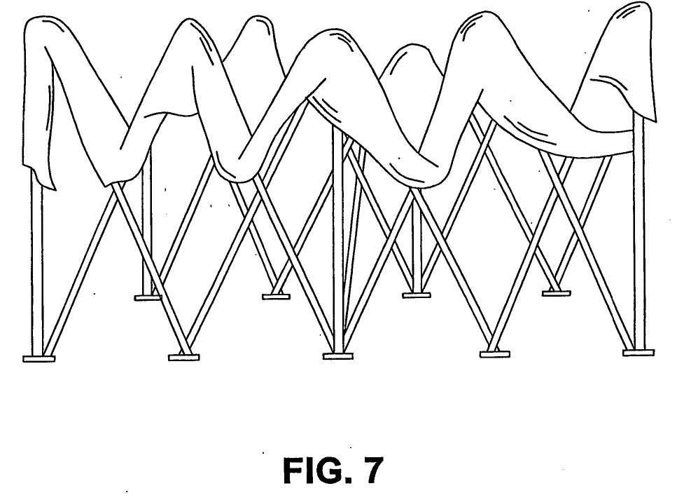

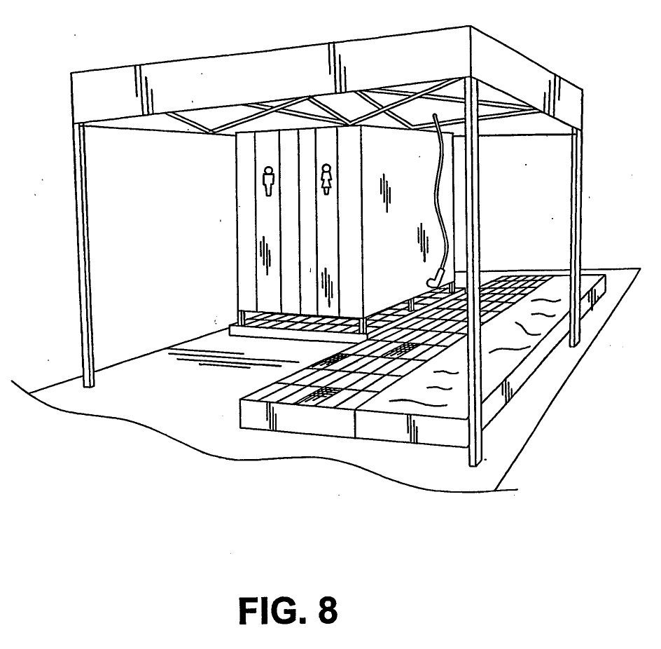

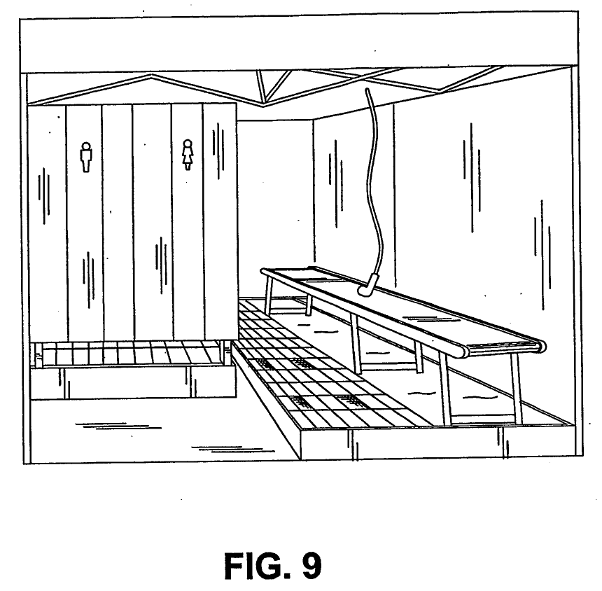

FIG. 6 is a photograph of a complete decontamination system mounted on a wheeled cart. Such a system could be used for example at the emergency receiving entrance of a hospital, the cart being stored in the hospital and wheeled outside to decontaminate casualties as they arrive at the hospital. The cart contains the shelter shown partially erected in FIG. 7, FIG. 8 shows the shower suite being installed on a drainage pan in the shelter, and FIG. 9 shows the completed decontamination system with the soft roller conveyor and drainage pan, with shower spray inside the shelter. The cart of FIG. 6 contains all components, except for the fresh water supply, which would be obtained from an adjacent building, or a separate sealed fresh water supply, and the grey water from the system could be retained in a suitable tank. The system of FIGS. 6-9 has been erected in fifteen minutes by a team of two, making the availability of decontamination to a medical facility on short notice.

Systems in accordance with the present invention, can be stored in suitable containers such as standard ISO shipping containers, on trailers, or in air freight containers for rapid deployment to hazardous sites as required.

Those skilled in the field of first response will recognize that variations of the system may be made to adapt to field conditions, and all such variations within the scope of the claims of this application are intended to by covered by this application.

1. A transportable mass personnel decontamination system comprising:
a) a transportation means carrying;
b) at least one individual three compartment processing cubicle, each processing cubicle having enclosure means for each compartment to provide modesty screening of personnel, the first compartment for disrobing, the second compartment containing a temperature and pressure controlled personnel operated shower with decontaminant dispenser, the third compartment providing a re-dress area for ambulatory personnel;
c) a monitoring area;
d) a fresh water storage tank;
e) a fresh water temperature and pressure control system; and
f) a grey water collection and storage tank.

2. The invention defined in claim 1 further including at least one soft conveyor litter patient processing line said line including stations for decontamination, washing, rinsing and drying litter patients.

3. The invention defined in claim 1, wherein said transportation means includes a road trailer, a semi-trailer, fork lift pallets, an ISO shipping container and an air cargo container.

4. The invention defined in claim 1 wherein each processing cubicle is formed of a collapsible light weight framing system having opaque plastic curtain material forming vertical sides, and four removable door flaps separating the first compartment from the exterior, the second compartment, from the first and third compartments, and the third compartment from the exterior.

5. The invention of claim 1, wherein the fresh water temperature and pressure control system includes pressurizing means and heating means, said heating means including an anti-scald control for limiting water temperature, and a decontaminant dispenser.

6. The invention of claim 5, wherein the water pressure is limited to less then 60 psi, and the temperature is maintained below scalding temperature.

7. The invention of claim 3 wherein said transportation system comprises a trailer with droppable sides forming ramps for entering and exiting the decontamination cubicles, said cubicles being arranged side by side transversely of the trailer, with all first compartments on the entry side of the trailer, said trailer being intended to be positioned on a line demarcating a contaminated zone and a clean zone, with the entry in the contamination zone.

8. The invention of claim 1 or 6 in which the grey water collection and storage tank is positioned in the contamination zone.

9. The invention of claim 1 in which the grey water tank comprises a collapsible onion tank, connected to a drainage system for collection of grey water from said showers.

10. The invention of claim 8 in which the drainage system includes grid materials in a drainage tray in said second compartments, and conduit means connecting said drainage tray to said onion tank.
